(19) United States
(12) Patent Application Publication
(10) Pub. No.: US 2025/0049613 A1
(43) Pub. Date: Feb. 13, 2025

(54) NEGATIVE PRESSURE WOUND THERAPY DRESSING WITH A SLITTED FOAM LAYER

(71) Applicant: KCI MANUFACTURING UNLIMITED COMPANY, Westmeath (IE)

(72) Inventors: Christoph SCHUELL, Saint Paul, MN (US); Audrey SHERMAN, Saint Paul, MN (US); Guido HITSCHMANN, Saint Paul, MN (US); Hironobu ISHIWATARI, Saint Paul, MN (US); Daniel W. DEKRUIF, Saint Paul, MN (US); James A. LUCKEMEYER, Saint Paul, MN (US)

(21) Appl. No.: 18/718,392

(22) PCT Filed: Nov. 17, 2022
(86) PCT No.: PCT/IB2022/061070

§ 371 Date: Jun. 10, 2024

Related U.S. Application Data
(60) Provisional application No. 63/291,280, filed on Dec. 17, 2021.

(51) Int. Cl. A61F 13/0203

(52) U.S. Cl. CPC A61F 13/0203

(57) ABSTRACT

Apparatuses, dressings, systems, and methods for treating a tissue site with negative pressure. The dressing includes a cover and a tissue interface including a manifold and a film layer. The manifold includes a plurality of slits. The plurality of slits include a first side wall and a second side wall that are deformable between a closed state and an open state. The film layer includes a plurality of fenestrations through a first surface and a second surface of the film layer that are aligned with the plurality of slits of the foam.

### CROSS-REFERENCE TO RELATED APPLICATIONS

This application is a U.S. National Stage Entry of PCT/IB2022/061070, filed Nov. 17, 2022, which claims the benefit of priority to U.S. Provisional Application No. 63/291,280, filed Dec. 17, 2021, each of which are incorporated herein by reference in their entirety.

### TECHNICAL FIELD

The invention set forth in the appended claims relates generally to tissue treatment systems and more particularly, but without limitation, to negative pressure wound therapy dressings including a slitted foam layer.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for negative pressure wound therapy dressings including a slitted foam layer in a negative-pressure therapy environment are set forth in the appended claims. Illustrative embodiments are also provided to enable a person skilled in the art to make and use the claimed subject matter.

In some example embodiments, a dressing for treating a tissue site with negative pressure is described. The dressing can include a cover, a manifold including a foam, and a film layer. The cover can include a first surface and a second surface. The manifold can include a first surface, a second surface, and a plurality of slits. The first surface of the manifold can be adjacent to the second surface of the cover. The plurality of slits can include a first side wall and a second side wall extending between the first surface and the second surface of the manifold that are deformable between a closed and an open state. The first side wall can be in contact with the second side wall in the closed state and can be separated from the second side wall in the open state. The film layer can include a first surface, a second surface, and a plurality of fenestrations through the first surface and the second surface. The first surface of the film layer can be adjacent to the second surface of the manifold.

In some example embodiments, the plurality of slits of the manifold are configured to deform from the closed state to the open state when the dressing is exposed to the negative pressure, an axial force or to a bending moment. The plurality of slits can define a slit opening through the foam between the first side wall and the second side wall in an open state. The plurality of slits can include a linear cut having a length. The first side wall can be positioned on an opposite side of the linear cut from the second side wall. For example, the first side wall can be positioned opposite from the second side wall across the slit opening of each of the plurality of slits.

In some example embodiments, one or more of the plurality of slits are deformable between the closed state and the open state independently of another of the plurality of slits. The plurality of fenestrations in the film layer can be configured to deform in alignment with the plurality of slits of the manifold when the dressing is exposed to the negative pressure. More generally, the plurality of fenestrations of the film layer and the plurality of slits in the manifold are aligned in both the open state and the closed state and are configured to deform in concert in response to the negative pressure. In some example embodiments, the plurality of fenestrations can each comprise a perimeter edge that can be positioned flush with the first side wall and the second side wall of the plurality of slits. In some embodiments, the plurality of fenestrations and the plurality of slits can have the same shape.

In some example embodiments, the dressing can further comprise a base layer adjacent to the second surface of the film layer. The base layer can include a central portion with a base layer opening and a peripheral portion including a plurality of apertures. The peripheral portion can surround the central portion and the plurality of apertures in the peripheral portion can be smaller than the base layer opening in the central portion. The base layer opening can be a single opening and can surround at least 90 percent of the plurality of fenestrations of the film layer. The plurality of fenestrations and at least a portion of the film layer can be exposed through the base layer opening. For example, the film layer can be configured to contact the tissue site through the base layer opening and the peripheral portion of the base layer can be configured to contact tissue surrounding the tissue site. In some example embodiments, the film layer can include a central portion including the plurality of fenestrations aligned with the foam and a peripheral portion extending beyond the foam and including a plurality of perforations surrounding the plurality of fenestrations.

In some example embodiments, the cover can include a margin with an adhesive that extends beyond the manifold. The margin of the cover can be configured to enclose the manifold between the base layer and the cover. The adhesive of the margin can be configured to extend through the plurality of apertures in the base layer to contact tissue surrounding the tissue site. The dressing can further include at least one handling bar configured to add rigidity to a portion of the dressing to enable a user to place the dressing at a tissue site.

A system for treating a tissue site with negative pressure is also described herein. Illustrative examples of the system can include a dressing and a negative-pressure source that can be fluidly coupled to the dressing. The dressing can include a cover, a manifold including a foam, and a film layer. The cover can include a first surface and a second surface. The manifold can include a first surface, a second surface, and a plurality of slits. The first surface of the manifold can be adjacent to the second surface of the cover. The plurality of slits can include a first side wall and a second side wall extending between the first surface and the second surface of the manifold that are deformable between a closed and an open state. The first side wall can be in contact with the second side wall in the closed state and can be separated from the second side wall in the open state. The film layer can include a first surface, a second surface, and a plurality of fenestrations through the first surface and the second surface. The first surface of the film layer can be adjacent to the second surface of the manifold.

A tissue interface for treating a tissue site with negative pressure is also described herein. Illustrative examples of the tissue interface can include a manifold layer and a film layer. The manifold layer can include a plurality of slits and each of the plurality of slits can include a first side wall and a second side wall. The film layer can be coupled to a surface of the manifold layer and can include a plurality of fenestrations. Each of the plurality of fenestrations can include a perimeter edge positioned flush with the first side wall and the second side wall of the plurality of slits.

A method of treating a tissue site is also described herein. Illustrative examples of the method can include applying a dressing to the tissue site, fluidly coupling a negative-pressure source to the dressing, and actuating the negative-pressure source to apply negative pressure to the dressing. The dressing can include a foam with a plurality of slits and a film layer positioned between the foam and the tissue site. The film layer can include a plurality of fenestrations configured to align with the plurality of slits of the foam. When actuating the negative-pressure source to apply negative pressure to the dressing, the plurality of slits of the foam can be deformed in concert with the plurality of fenestrations of the film layer when the negative pressure is applied to the dressing.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a block diagram of an example embodiment of a therapy system that can provide negative pressure treatment in accordance with this specification; and

FIG. 2 is a cut-away view of an illustrative embodiment of the therapy system of FIG. 1 depicting an illustrative example embodiment of a dressing interface and a dressing deployed at a tissue site;

FIG. 3 is an exploded view of the dressing of FIG. 2, depicted with an illustrative example embodiment of a release liner for protecting the dressing prior to application at a tissue site;

FIG. 4 is a cut-away view of the dressing of FIG. 2;

FIG. 5 is a perspective view of an illustrative example embodiment of a manifold and a film layer depicted in the dressing of FIG. 2;

FIG. 6A is a perspective view of another illustrative example embodiment of a manifold that can be used with the dressing of FIG. 2;

FIG. 6B is a perspective view of the manifold of FIG. 6A when a force is applied to the manifold;

FIG. 6C is a perspective view of the manifold of FIG. 6A when a force is applied to the manifold;

FIG. 6D is a perspective view of the manifold of FIG. 6A when a force is applied to the manifold; and

FIG. 7 is an exploded view of another illustrative example embodiment of a dressing that can be used with the therapy system of FIG. 1;

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

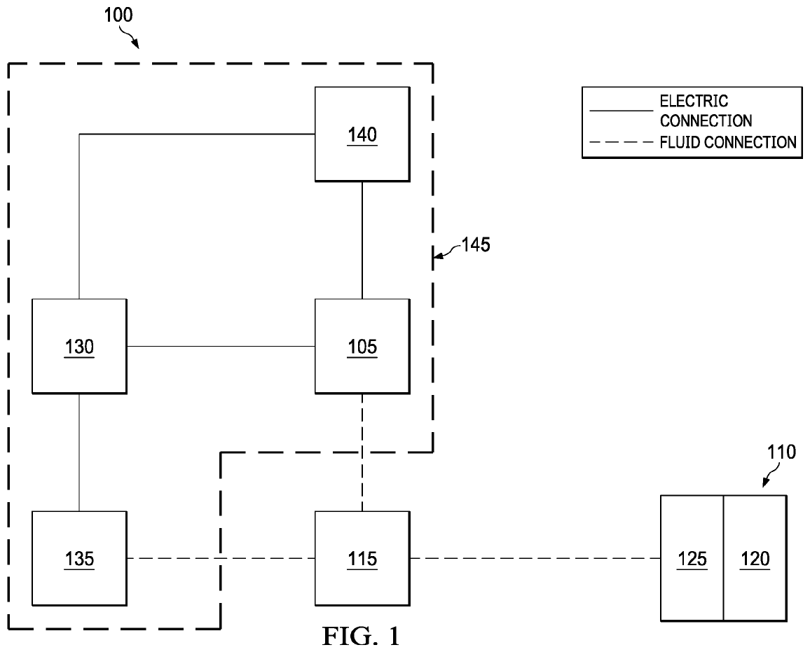

FIG. 1 is a block diagram of an example embodiment of a therapy system 100 that can provide negative-pressure therapy to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

Referring to FIG. 1, the therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Texas.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130 and other components into a therapy unit 145.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, can be apparatuses operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. The signals from the first sensor 135 and the second sensor 140 may be suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials; polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S.A., Colombes, France; and Inspire 2301 and Inspire 2327 polyurethane films, commercially available from Expopack Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The process of reducing pressure may be described illustratively herein as “delivering,” “distributing,” or “generating” negative pressure, for example. In general, exudate and other fluid flow toward lower pressure along a fluid path. Thus, the term “downstream” typically implies a location in a fluid path relatively closer to a source of negative pressure or further away from a source of positive pressure. Conversely, the term “upstream” implies a location relatively further away from a source of negative pressure or closer to a source of positive pressure. However, the fluid path may also be reversed in some applications, such as by substituting a positive-pressure source for a negative-pressure source; and therefore, these descriptive terms should not be construed as limiting.

Negative pressure applied to the tissue site through the tissue interface 120 in the sealed therapeutic environment can induce macro-strain and micro-strain in the tissue site. Negative pressure can also remove exudate and other fluid from a tissue site, which can be collected in the container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, the controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

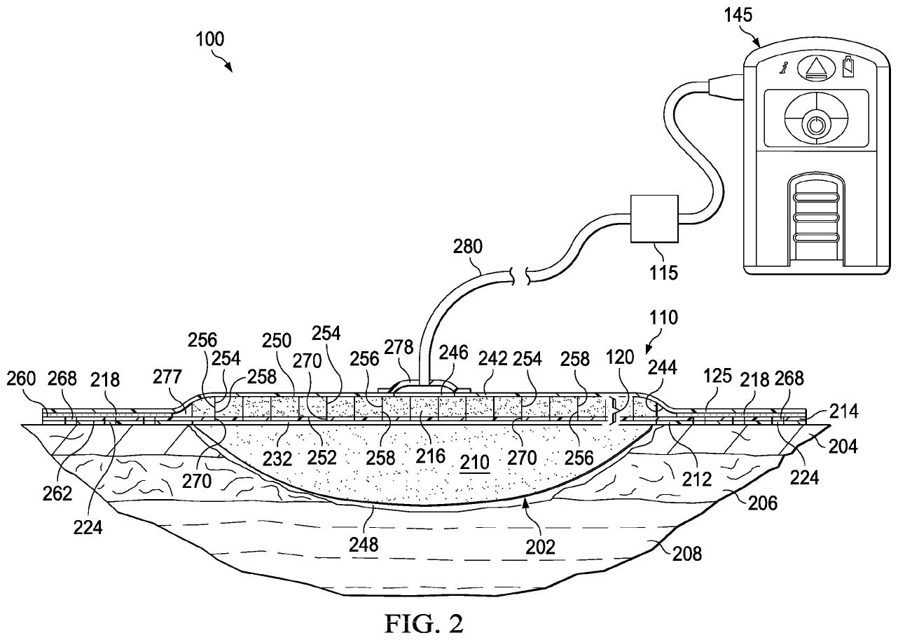

FIG. 2 depicts an example embodiment of the therapy system 100 for treating a tissue site 202 of a patient. The tissue site 202 may extend through or otherwise involve an epidermis 204, a dermis 206, and a subcutaneous tissue 208. The tissue site 202 may be a sub-surface tissue site as depicted in FIG. 2 that extends below the surface of the epidermis 204. Further, the tissue site 202 may be a surface tissue site (not shown) that predominantly resides on the surface of the epidermis 204, such as, for example, an incision. The therapy system 100 may provide therapy to, for example, the epidermis 204, the dermis 206, and the subcutaneous tissue 208, regardless of the positioning of the therapy system 100 or the type of tissue site. The therapy system 100 may also be utilized without limitation at other tissue sites.

The therapy system 100 may include the dressing 110, the container 115, and the therapy unit 145 that may include the negative-pressure source 105. Further, the therapy system 100 may include a filler material 210 as an optional component of the therapy system 100. The filler material 210 may be omitted for different types of tissue sites or different types of therapy using negative pressure, such as, for example, epithelialization. If equipped, the filler material 210 may be adapted to be positioned proximate to or adjacent to the tissue site 202, such as, for example, by cutting or otherwise shaping the filler material 210 in any suitable manner to fit the tissue site 202 and to fill a space between the tissue site 202 and the dressing 110. Similar to the tissue interface 120, the filler material 210 may be constructed of the manifold materials described herein and may be adapted to be positioned in fluid communication with the tissue site 202 to distribute negative pressure to the tissue site 202. In some embodiments, the filler material 210 may be positioned in direct contact with the tissue site 202 and between the tissue site 202 and the dressing 110. If the filler material 210 is omitted, the tissue interface 120 of the dressing 110 may be positioned in direct contact with the tissue site 202.

Continuing with FIG. 2, the dressing 110 may be adapted to provide or distribute negative pressure from the negative-pressure source 105 of the therapy unit 145 to the tissue site 202 directly or through the filler material 210, if equipped. Further, FIG. 2 illustrates additional features that may be associated with some example embodiments of the tissue interface 120 of the dressing 110. For example, the tissue interface 120 of the dressing 110 may include an optional base layer 212, a film layer 214, and a manifold 216. The manifold 216 can be or can include a foam and may be referred to as a manifold layer. An adhesive layer such as an adhesive 218 may be configured to be positioned between the cover 125 and a periphery of the tissue site 202 to secure the dressing 110 relative to the tissue site 202. Components of the dressing 110 may be added or removed to suit a particular application.

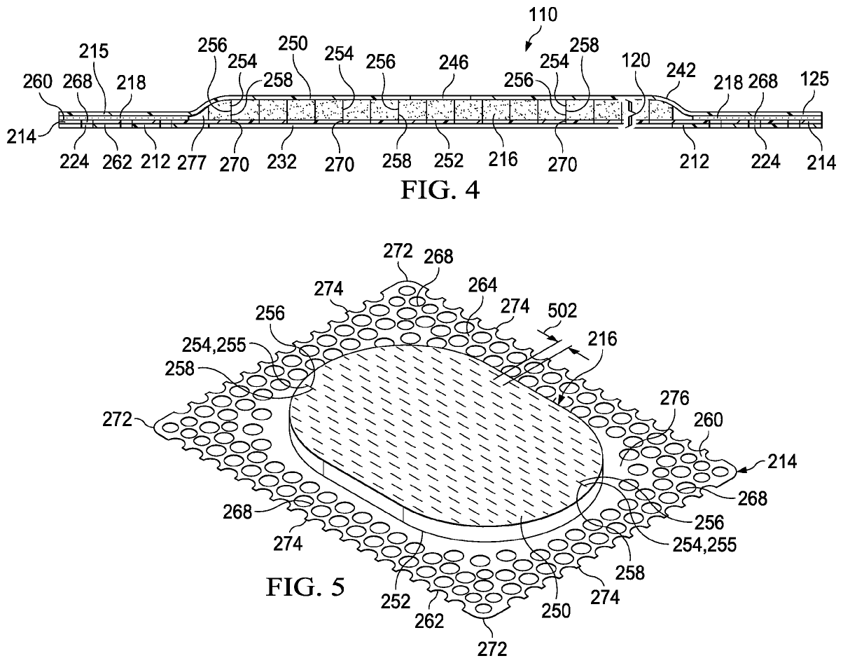

Referring to FIGS. 2-4, the base layer 212 may have a periphery 220 surrounding a central portion 222, and a plurality of apertures 224 disposed through the periphery 220. The base layer 212 may also have corners 226 and edges 228. The corners 226 and the edges 228 may be part of the periphery 220. One of the edges 228 may meet another of the edges 228 to define one of the corners 226. Further, the base layer 212 may have a border 230 substantially surrounding the central portion 222 and positioned between the central portion 222 and the periphery 220. The border 230 may be free of the apertures 224 and may surround a base layer opening 232 through the central portion 222 of the base layer 212. The base layer opening 232 may be a single opening and may prevent the base layer 212 from contacting the tissue site 202. Each aperture 224 of the plurality of apertures 224 may be smaller than the base layer opening 232. The base layer 212 may couple to tissue surrounding the tissue site 202 such that the base layer opening 232 of the central portion 222 of the base layer 212 is positioned adjacent to or proximate to the tissue site 202, and the periphery 220 of the base layer 212 is positioned adjacent to or proximate to tissue surrounding the tissue site 202. In this manner, the periphery 220 of the base layer 212 may surround the tissue site 202.

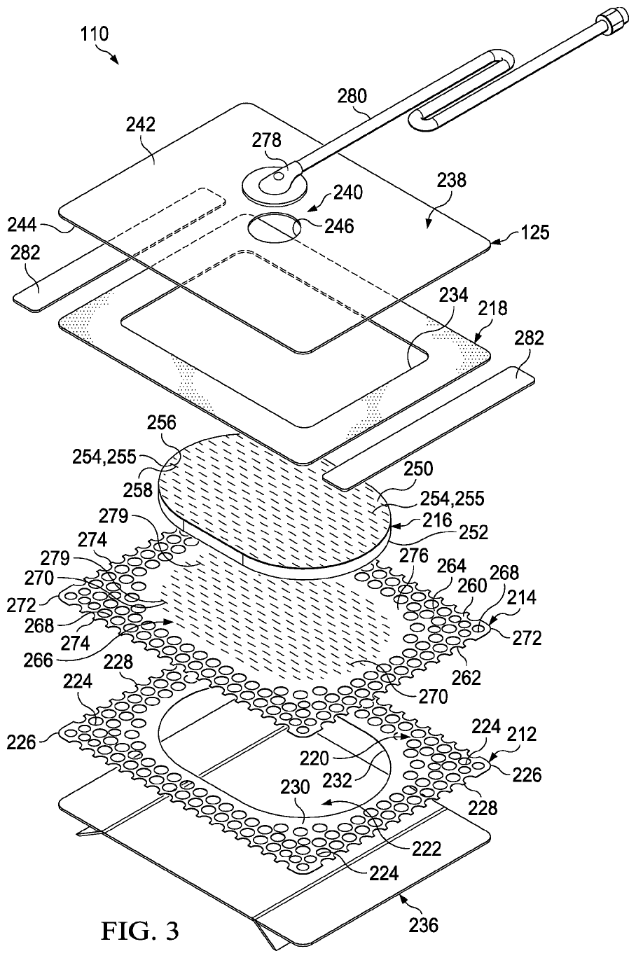

The apertures 224 in the base layer 212 may have another shape, such as, for example, circles, squares, stars, ovals, polygons, slits, complex curves, rectilinear shapes, triangles, or other shapes. The apertures 224 may be formed by cutting, by application of local RF energy, or other suitable techniques for forming an opening. As shown in FIG. 3, each of the apertures 224 of the plurality of apertures 224 may be substantially circular in shape, having a diameter and an area. The area of each of the apertures 224 may refer to an open space or open area defining each of the apertures 224. The diameter of each of the apertures 224 may define the area of each of the apertures 224. For example, the area of one of the apertures 224 may be defined by multiplying the square of half the diameter of the aperture 224 by the value 3.14. Thus, the following equation may define the area of one of the apertures 224: Area=3.14*(diameter/2)\hat{ }2. The area of the apertures 224 described in the illustrative embodiments herein may be substantially similar to the area in other embodiments (not shown) for the apertures 224 that may have non-circular shapes. The diameter of each of the apertures 224 may be substantially the same, or each of the diameters may vary depending, for example, on the position of the aperture 224 in the base layer 212. Further, the diameter of each of the apertures 224 may be between about 1 millimeter to about 50 millimeters. In some embodiments, the diameter of each of the apertures 224 may be between about 1 millimeter to about 20 millimeters. The apertures 224 may have a uniform pattern or may be randomly distributed on the base layer 212. The size and configuration of the apertures 224 may be designed to control the adherence of the dressing 110 to the epidermis 204 as described below.

The base layer 212 may be a soft, pliable material suitable for providing a fluid seal with the tissue site 202 as described herein. For example, the base layer 212 may comprise a silicone gel, a soft silicone, hydrocolloid, hydrogel, polyurethane gel, polyolefin gel, hydrogenated styrenic copolymer gels, a foamed gel, a soft closed cell foam such as polyurethanes and polyolefins coated with an adhesive described below, polyurethane, polyolefin, or hydrogenated styrenic copolymers. In some embodiments, the base layer 212 may be a trilaminate material including a film layer, a silicone gel coupled to a surface of the film layer proximate to the tissue site 202, and an adhesive layer coupled to a surface of the film layer opposite the silicone gel. The base layer 212 may have a thickness between about 500 microns (μm) and about 1000 microns (μm). In some embodiments, the base layer 212 has a stiffness between about 5 Shore OO and about 90 Shore OO. The base layer 212 may be comprised of hydrophobic or hydrophilic materials.

In some embodiments (not shown), the base layer 212 may be a hydrophobic-coated material. For example, the base layer 212 may be formed by coating a spaced material, such as, for example, woven, nonwoven, molded, or extruded mesh with a hydrophobic material. The hydrophobic material for the coating may be a soft silicone, for example. In this manner, the adhesive 218 may extend through openings in the spaced material analogous to the apertures 224 as described below.

The adhesive 218 may be in fluid communication with the apertures 224 in at least the periphery 220 of the base layer 212. In this manner, the adhesive 218 may be in fluid communication with the tissue surrounding the tissue site 202 through the apertures 224 in the base layer 212. As described below, the adhesive 218 may extend or be pressed through the plurality of apertures 224 to contact the epidermis 204 for securing the dressing 110 to, for example, the tissue surrounding the tissue site 202. The apertures 224 may provide sufficient contact of the adhesive 218 to the epidermis 204 to secure the dressing 110 about the tissue site 202. However, the configuration of the apertures 224 and the adhesive 218 may permit release and repositioning of the dressing 110 about the tissue site 202.

The adhesive 218 may be a medically-acceptable adhesive. The adhesive 218 may also be flowable. For example, the adhesive 218 may comprise an acrylic adhesive, rubber adhesive, high-tack silicone adhesive, polyurethane, or other adhesive substance. In some embodiments, the adhesive 218 may be a pressure-sensitive adhesive comprising an acrylic adhesive with coating weight of 15 grams/m² (gsm) to 70 grams/m² (gsm). The adhesive 218 may be a layer having an opening 234. The opening 234 of the adhesive 218 may ensure that the adhesive 218 does not come in contact with the central portion 222 of the base layer 212. The opening 234 may be rectangular as shown in FIG. 3. In other embodiments, the opening 234 may be a different size or shape but may still isolate the adhesive 218 from the central portion 222 of the base layer 212. In some embodiments, the layer of the adhesive 218 may be continuous or discontinuous. Discontinuities in the adhesive 218 may be provided by apertures (not shown) in the adhesive 218. The apertures in the adhesive 218 may be formed after application of the adhesive 218 or by coating the adhesive 218 in patterns on a carrier layer, such as, for example, a side of the cover 125 adapted to face the epidermis 204. Further, the apertures in the adhesive 218 may be sized to control the amount of the adhesive 218 extending through the apertures 224 in the base layer 212 to reach the epidermis 204. The apertures in the adhesive 218 may also be sized to enhance the Moisture Vapor Transfer Rate (MVTR) of the dressing 110.

Factors that may be utilized to control the adhesion strength of the dressing 110 may include the diameter and number of the apertures 224 in the base layer 212, the thickness of the base layer 212, the thickness and amount of the adhesive 218, and the tackiness of the adhesive 218. An increase in the amount of the adhesive 218 extending through the apertures 224 generally corresponds to an increase in the adhesion strength of the dressing 110. A decrease in the thickness of the base layer 212 generally corresponds to an increase in the amount of adhesive 218 extending through the apertures 224. Thus, the diameter and configuration of the apertures 224, the thickness of the base layer 212, and the amount and tackiness of the adhesive utilized may be varied to provide a desired adhesion strength for the dressing 110. For example, the thickness of the base layer 212 may be about 200 microns, the layer of adhesive 218 may have a thickness of about 30 microns and a tackiness of 2000 grams per 25 centimeter wide strip, and the diameter of the apertures 224 in the base layer 212 may be about 10 millimeters.

In some embodiments, the tackiness of the adhesive 218 may vary in different locations of the base layer 212. For example, some of the apertures 224 of the base layer 212 may be larger than other apertures 224 of the base layer 212. For example, in some embodiments, apertures 224 in the corners 226 of the base layer 212 may be smaller than apertures 224 along the edges 228 of the base layer 212. In locations of the base layer 212 where the apertures 224 are comparatively large, the adhesive 218 may have a lower tackiness than other locations of the base layer 212 where the apertures 224 are smaller. In this manner, locations of the base layer 212 having larger apertures 224 and adhesive 218 with lower tackiness may have an adhesion strength comparable to locations having smaller apertures 224 and adhesive 218 with higher tackiness.

Clinical studies have shown that the configuration described herein for the base layer 212 and the adhesive 218 may reduce the occurrence of blistering, erythema, and leakage when in use. Such a configuration may provide, for example, increased patient comfort and increased durability of the dressing 110.

Referring to the embodiment of FIG. 3, a release liner 236 may be attached to or positioned adjacent to the base layer 212 to protect the adhesive 218 prior to application of the dressing 110 to the tissue site 202. In some embodiments, the release liner 236 may be a one-piece liner. In other embodiments, the release liner 236 may be a two-piece liner with overlap to ensure that the base layer 212 is covered by the release liner 236 prior to deployment of the dressing 110 at the tissue site 202. Prior to application of the dressing 110 to the tissue site 202, the base layer 212 may be positioned between the cover 125 and the release liner 236. Removal of the release liner 236 may expose the base layer 212 and the adhesive 218 for application of the dressing 110 to the tissue site 202. The release liner 236 may also provide stiffness to assist with, for example, deployment of the dressing 110. The release liner 236 may be, for example, a casting paper, a film, or polyethylene. Further, the release liner 236 may be a polyester material such as polyethylene terephthalate (PET), or similar polar semi-crystalline polymer. The use of a polar semi-crystalline polymer for the release liner 236 may substantially preclude wrinkling or other deformation of the dressing 110. For example, the polar semi-crystalline polymer may be highly orientated and resistant to softening, swelling, or other deformation that may occur when brought into contact with components of the dressing 110, or when subjected to temperature or environmental variations, or sterilization. Further, a release agent may be disposed on a side of the release liner 236 that is configured to contact the base layer 212. For example, the release agent may be a silicone coating and may have a release factor suitable to facilitate removal of the release liner 236 by hand and without damaging or deforming the dressing 110. In some embodiments, the release agent may be fluorosilicone. In other embodiments, the release liner 236 may be uncoated or otherwise used without a release agent.

Continuing with FIGS. 2-4, the cover 125 may be substantially as described above with reference to FIG. 1. The cover 125 may have a margin or a periphery 238 and a central portion 240. The cover 125 may also have a first surface 242 and a second surface 244 opposite the first surface 242. The cover 125 may additionally include an aperture 246. The aperture 246 may be an opening or a hole through the cover 125. In some embodiments, the aperture 246 may be located substantially centered on the cover 125. The aperture 246 may be configured to allow fluid communication from the first surface 242 of the cover 125 through the dressing 110. The periphery 238 of the cover 125 may be positioned proximate to the periphery 220 of the base layer 212 and the central portion 240 of the cover 125 may be substantially aligned with the central portion 222 of the base layer 212.

The adhesive 218 may be positioned at least between the periphery 238 of the cover 125 and the periphery 220 of the base layer 212. The cover 125 may cover the tissue site 202 and the tissue interface 120 to provide a fluid seal and a sealed space 248 between the tissue site 202 and the cover 125 of the dressing 110. Further, the cover 125 may cover other tissue, such as a portion of the epidermis 204, surrounding the tissue site 202 to provide the fluid seal between the cover 125 and the tissue site 202. In some embodiments, a portion of the periphery 238 of the cover 125 may extend beyond the periphery 220 of the base layer 212 and into direct contact with tissue surrounding the tissue site 202. In other embodiments, the periphery 238 of the cover 125, for example, may be positioned in contact with tissue surrounding the tissue site 202 to provide the sealed space 248 without the base layer 212. Thus, the adhesive 218 may also be positioned at least between the periphery 238 of the cover 125 and tissue, such as the epidermis 204, surrounding the tissue site 202. The adhesive 218 may be disposed on a surface of the cover 125 adapted to face the tissue site 202 and the base layer 212.

The manifold 216 may be positioned between the cover 125 and the film layer 214. The manifold 216 may have a first surface 250 and a second surface 252 opposite the first surface 250. The manifold 216 may comprise or consist essentially of a means for distributing fluid relative to the tissue site 202. For example, the manifold 216 may be adapted to receive negative pressure from the negative-pressure source 105 and distribute negative pressure through the manifold 216, which may have the effect of collecting fluid from the tissue site 202 and drawing the fluid toward the negative-pressure source 105. The manifold 216 may further include a plurality of slits 254 disposed through the manifold 216 from the first surface 250 to the second surface 252. The plurality of slits 254 may be configured to help distribute negative pressure through the manifold 216. In some embodiments, the plurality of slits 254 may enable negative pressure to be distributed relatively evenly to the tissue site 202 through the manifold 216 while reducing the possibility of tissue ingrowth from the tissue site 202 to the dressing 110.

Each slit 254 of the plurality of slits 254 may comprise a linear slit or a linear cut 255 extending from the first surface 250 of the manifold 216 to the second surface 252 of the manifold 216. In other embodiments, the plurality of slits 254 may be a different shape but may still extend from the first surface 250 of the manifold 216 to the second surface 252 of the manifold 216. The plurality of slits 254 may be formed by cutting, by application of local RF energy, by die cutting, by knife cutting, by discontinuous slitting, or other suitable techniques for forming a slit through the manifold 216. As defined herein, the slits or cuts forming the slits 254 are distinguishable from an aperture or other opening in that portions of material on opposing sides of the slits 254 are severed or cut, without removing the material, to form the slits 254. Therefore, the plurality of slits 254 may include a first side wall 256 and a second side wall 258 that may both extend between the first surface 250 and the second surface 252 of the manifold 216. The first side wall 256 may be positioned on an opposite side of the linear cut 255 from the second side wall 258.

The first side wall 256 and the second side wall 258 may be deformable between a closed state and an open state. In the closed state in which the manifold 216 is relaxed or unstressed, the first side wall 256 may be in contact with the second side wall 258. In the open state in which the manifold 216 may be subjected to a stress or a force, the first side wall 256 may be separated from the second side wall 258. In some embodiments, the plurality of slits 254 may be deformable or moveable from the closed state to the open state when the dressing 110 is exposed to a negative pressure such as a negative pressure from the negative-pressure source 105. In other embodiments, the plurality of slits 254 may be deformable or moveable from the closed state to the open state when the dressing 110 is exposed to a force or bending moment. Each slit 254 of the plurality of slits 254 may be deformable or moveable between the closed state and the open state independently from another slit 254 of the plurality of slits 254.

In some illustrative embodiments, the manifold 216 may comprise a plurality of pathways, which can be interconnected to improve distribution or collection of fluids. In some illustrative embodiments, the manifold 216 may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, the manifold 216 may additionally or alternatively comprise projections that form interconnected fluid pathways. For example, the manifold 216 may be molded to provide surface projections that define interconnected fluid pathways.

In some embodiments, the manifold 216 may comprise or consist essentially of a foam such as a reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the manifold 216 may also vary according to needs of a prescribed therapy. The 25% compression load deflection of the manifold 216 may be at least 0.35 pounds per square inch, and the 65% compression load deflection may be at least 0.43 pounds per square inch. In some embodiments, the tensile strength of the manifold 216 may be at least 10 pounds per square inch. The manifold 216 may have a tear strength of at least 2.5 pounds per inch. In some embodiments, the manifold 216 may be foam comprised of polyols such as polyester or polyether, isocyanate such as toluene diisocyanate, and polymerization modifiers such as amines and tin compounds. In some examples, the manifold 216 may be reticulated polyurethane foam such as found in GRANUFOAM™ dressing or V.A.C. VERAFLO™ dressing, both available from Kinetic Concepts, Inc. of San Antonio, Texas. In other embodiments, the manifold may be a thermoplastic polyurethane foam, a viscoelastic polyurethane foam or a laminate of one or more of the polyurethane foams.

The thickness of the manifold 216 may also vary according to needs of a prescribed therapy. For example, the thickness of the manifold 216 may be decreased to reduce tension on peripheral tissue of the tissue site 202. The thickness of the manifold 216 can also affect the conformability of the manifold 216. In some embodiments, a thickness in a range of about 5 millimeters to 10 millimeters may be suitable.

In some exemplary embodiments, the manifold 216 may be hydrophilic. In an example in which the manifold 216 may be hydrophilic, the manifold 216 may also wick fluid away from a tissue site, while continuing to distribute negative pressure to the tissue site 202. The wicking properties of the manifold 216 may draw fluid away from the tissue site 202 by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the manifold 216 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The manifold 216 may further serve as a scaffold for new cell-growth, or a scaffold material may be used in conjunction with the manifold 216 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

The film layer 214 may be disposed between the manifold 216 and the base layer 212, if equipped. In embodiments without the base layer 212, the film layer 214 may be disposed between the manifold 216 and the tissue site 202. The film layer 214 may have a first surface 260 and a second surface 262 opposite the first surface 260. The first surface 260 of the film layer 214 may be adjacent to the second surface 252 of the manifold 216. The film layer 214 may have a periphery 264 surrounding a central portion 266, a plurality of perforations 268 disposed through the periphery 264, and a plurality of fenestrations 270 disposed through the central portion 266. The plurality of perforations 268 may be larger than the plurality of fenestrations 270. The film layer 214 may also have corners 272 and edges 274. The corners 272 and the edges 274 may be part of the periphery 264. One of the edges 274 may meet another of the edges 274 to define one of the corners 272. Further, the film layer 214 may have a border 276 substantially surrounding the central portion 266 and positioned between the central portion 266 and the periphery 264. The border 276 may be free of the plurality of perforations 268 and the plurality of fenestrations 270.

The film layer 214 may be substantially the same shape as the base layer 212 such that the periphery 264 of the film layer 214 aligns with the periphery 220 of the base layer 212 and the central portion 266 of the film layer 214 aligns with the central portion 222 of the base layer 212. The periphery 264 of the film layer 214 may further align with the periphery 238 of the cover 125. The periphery 264 of the film layer 214 may be positioned proximate to the periphery 238 of the cover 125 such that the central portion 240 of the cover 125 and the central portion 266 of the film layer 214 define an enclosure 277. The periphery 238 of the cover 125 may enclose the manifold 216 between the film layer 214 and the cover 125. The enclosure 277 may be configured to allow the manifold 216 to deform when exposed to a negative pressure, to an axial force, to a bending moment, or to another force. The manifold 216 may be disposed within the enclosure 277 such that it is isolated from the adhesive 218.

The plurality of fenestrations 270 may be substantially aligned with the base layer opening 232 such that the film layer 214 is configured to contact the tissue site 202 through the base layer opening 232. More specifically, the plurality of fenestrations 270 and at least a portion of the film layer 214 may be exposed to the tissue site through the base layer opening 232. In some embodiments, the base layer opening 232 may surround at least 90% of the plurality of fenestrations 270. In other embodiments, the base layer opening 232 may surround more or less than 90% of the plurality of fenestrations 270 and may allow fluid communication from the tissue site 202 to the plurality of fenestrations 270. The plurality of perforations 268 of the film layer 214 may align with the apertures 224 of the base layer 212 to allow the adhesive 218 to contact the tissue surrounding the tissue site 202 substantially as described above. The central portion 266 of the film layer 214 may be configured to be in fluid communication with the tissue site 202 through the base layer opening 232 of the base layer 212.

The plurality of perforations 268 in the film layer 214 may have another shape, such as, for example, circles, squares, stars, ovals, polygons, slits, complex curves, rectilinear shapes, triangles, or other shapes that may be aligned with the shape of the apertures 224 of the base layer 212, if equipped. The plurality of perforations 268 may be formed by cutting, by application of local RF energy, or another suitable technique for forming an opening. As shown FIG. 3, each perforation 268 of the plurality of perforations 268 may be substantially circular in shape, having a diameter and an area. The area of each perforation 268 of the plurality of perforations 268 may refer to an open space or open area defining each perforation 268 of the plurality of perforations 268. The diameter of each perforation 268 of the plurality of perforations 268 may define the area of each perforation 268 of the plurality of perforations 268. For example, the area of one of the perforations 268 of the plurality of perforations 268 may be defined by multiplying the square of half the diameter of the perforation 268 by the value 3.14. Thus, the following equation may define the area of one of the perforations 268: Area=3.14*(diameter/2)\hat{ }2. The area of the perforations 268 described in the illustrative embodiments herein may be substantially similar to the area in other embodiments (not shown) for the perforations 268 that may have non-circular shapes. The diameter of each perforation 268 of the plurality of perforations 268 may be substantially the same, or each of the diameters may vary depending, for example, on the position of the plurality of perforations 268 in the film layer 214. Further, the diameter of each perforation 268 of the plurality of perforations 268 may be between about 1 millimeter to about 50 millimeters. In some embodiments, the diameter of each perforation 268 of the plurality of perforations 268 may be between about 1 millimeter to about 20 millimeters. The plurality of perforations 268 may have a uniform pattern or may be randomly distributed on the film layer 214. The size and configuration of the plurality of perforations 268 may be designed to control the adherence of the dressing 110 to the epidermis 204.

The central portion 266 of the film layer 214 may include the plurality of fenestrations 270. Each fenestration 270 of the plurality of fenestrations 270 may be a slit or a cut through the film layer 214 from the first surface 260 of the film layer 214 to the second surface 262 of the film layer 214. The plurality of fenestrations 270 may be formed by cutting, by application of local RF energy, by die cutting, by knife cutting, by discontinuous slitting, or other suitable techniques for forming a slit through the film layer 214. Similar to the slits or cuts forming the slits 254, the slits or cuts forming the plurality of fenestrations 270 are distinguishable from an aperture or other opening in that portions of material on opposing sides of the fenestrations 270 are severed or cut, without removing the material, to form the fenestrations 270. In some embodiments, the film layer 214 may be coupled to the manifold 216 before the plurality of fenestrations 270 are formed in the film layer 214 and before the plurality of slits 254 are formed in the manifold 216. In some embodiments, the film layer 214 may be coupled to the manifold 216 and the plurality of fenestrations 270 and the plurality of slits 254 may be formed at the same time. By forming the plurality of fenestrations 270 and the plurality of slits 254 together, the alignment of the plurality of fenestrations 270 and the plurality of slits 254 may be ensured. In other embodiments the plurality of fenestrations 270 and the plurality of slits 254 may be formed in the respective layers separately and then the film layer 214 may be coupled to the manifold 216 so that the plurality of fenestrations 270 and the plurality of slits 254 are aligned. As such, the plurality of slits 254 and the plurality of fenestrations 270 may have the same shape.

The plurality of fenestrations 270 may be configured to deform when the dressing 110 is exposed to negative pressure such as negative pressure from the negative-pressure source 105. In some embodiments, the plurality of fenestrations 270 may be configured to deform when the dressing 110 is stretched or when the dressing 110 is exposed to an axial force or a bending moment. The plurality of fenestrations 270 of the film layer 214 may be aligned with the plurality of slits 254 of the manifold 216. Each fenestration 270 of the plurality of fenestrations 270 may include a perimeter edge defined by or around opposing edges 279 of each of the plurality of fenestrations 270. Although the opposing edges 279 are illustrated in FIGS. 2-5 as a linear cut, the opposing edges 279 are moveable or deformable between a closed state and an open state similar or analogous to the first side wall 256 and the second side wall 258 of the plurality of slits 254. The opposing edges 279 of each fenestration 270 of the plurality of fenestrations 270 may be positioned flush with the first side wall 256 and the second side wall 258 of the plurality of slits 254. Each fenestration 270 of the plurality of fenestrations 270 may be configured to deform in alignment with a corresponding slit 254 of the plurality of slits 254.

The film layer 214 may be comprised of a liquid-impermeable film. In some embodiments, the film layer 214 may comprise one or more of the following materials: hydrophilic polyurethane; cellulosics; hydrophilic polyamides; polyvinyl alcohol; polyvinyl pyrrolidone; hydrophilic acrylics; hydrophilic silicone elastomers; an INSPIRE 2301 material from Expopack Advanced Coatings of Wrexham, United Kingdom having, for example, an MVTR (inverted cup technique) of 14400 g/m²/24 hours and a thickness of about 30 microns; a thin, uncoated polymer drape; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; polyurethane (PU); EVA film; co-polyester; silicones; a silicone drape; a 3M Tegaderm® drape; a polyurethane (PU) drape such as one available from Avery Dennison Corporation of Pasadena, California; polyether block polyamide copolymer (PEBAX), for example, from Arkema, France; Expopack 2327; or other appropriate material.

In some embodiments, the film layer 214 may be a flexible, breathable film, membrane, or sheet having a high MVTR of, for example, at least about 300 g/m² per 24 hours. In other embodiments, a low or no vapor transfer drape might be used. The film layer 214 may comprise a range of medically suitable films having a thickness between about 15 microns (μm) to about 50 microns (μm). In other embodiments, the film layer 214 may be a non-breathable film, membrane, or sheet that may be substantially vapor and liquid impermeable. In some embodiments, the film layer 214 and the manifold 216 may be comprised of an elastic material to enable the film layer 214 and the manifold 216 to deform in alignment with each other. In some embodiments, the first surface 260 of the film layer 214 may be coated with an adhesive. The periphery 264 may contain the adhesive, if equipped, and the central portion 266 may be free from the adhesive. The adhesive may be similar to the adhesive 218 as described above.

Continuing with FIGS. 2 and 3, a dressing interface 278 may be configured to fluidly couple the dressing 110 to the container 115 and the therapy unit 145. The therapy system 100 may further include a fluid conductor or a conduit 280. The dressing interface 278 may be substantially as described above with reference to FIG. 1. In some embodiments, the dressing interface 278 may be an elbow connector which can be placed over the aperture 246 in the cover 125 to provide a fluid path between the conduit 280 and the tissue interface 120. The conduit 280 may be a flexible tube, which can be fluidly coupled on one end to the dressing interface 278. The conduit 280 may be coupled to the container 115 or the therapy unit 145 with an end opposite the end that is coupled to the dressing interface 278.

Referring to FIG. 3, in some embodiments, handling bars 282 may be optionally included in the dressing 110. In some embodiments, the handling bars 282 may be disposed between the cover 125 and the adhesive 218. In other embodiments, the handling bars 282 may be disposed between the adhesive 218 and the film layer 214. In still other embodiments, the handling bars 282 may be disposed in a different location of the dressing 110. The handling bars 282 may be configured to enable a user or a health care provider to place the dressing 110 at the tissue site 202. The handling bars 282 may prevent the user or health care provider from contacting the adhesive 218 while applying the dressing 110 to the tissue site 202. In some embodiments, the handling bars 282 may be configured to be removed from the dressing 110 after the dressing 110 is positioned at the tissue site 202 or may be removed with the release liner 236 as the dressing 110 is being placed at the tissue site 202. In other embodiments, the handling bars 282 may be partially or fully coated with an adhesive to enable the handling bars to lay flat at the tissue surrounding the tissue site 202 after the dressing 110 is positioned at the tissue site 202. In some embodiments, the handling bars 282 may be comprised of polycoated paper or another material that is strong enough to place the dressing 110 in place at the tissue site 202.

FIG. 5 is a perspective view of the film layer 214 and the manifold 216 of the dressing 110. The second surface 252 of the manifold 216 may be coupled to the first surface 260 of the film layer 214. The manifold 216 may be coupled to the central portion 266 of the film layer 214 such that the plurality of slits 254 of the manifold 216 are aligned with the plurality of fenestrations 270 of the film layer 214. At least a portion of the border 276 of the film layer 214 may be in contact with the manifold 216 such that the manifold 216 is in contact with the entirety of the central portion 266 of the film layer 214. Each slit 254 of the plurality of slits 254 may have a length 502. Each fenestration 270 of the plurality of fenestrations 270 may have a length equal to the length 502 so that the perimeter edge or opposing edges 279 of each fenestration 270 of the plurality of fenestrations 270 may be positioned flush with the first side wall 256 and the second side wall 258 of the corresponding slit 254 of the plurality of slits 254. Thus, each slit 254 of the plurality of slits 254 may be the same size and shape as a corresponding fenestration 270 of the plurality of fenestrations 270.

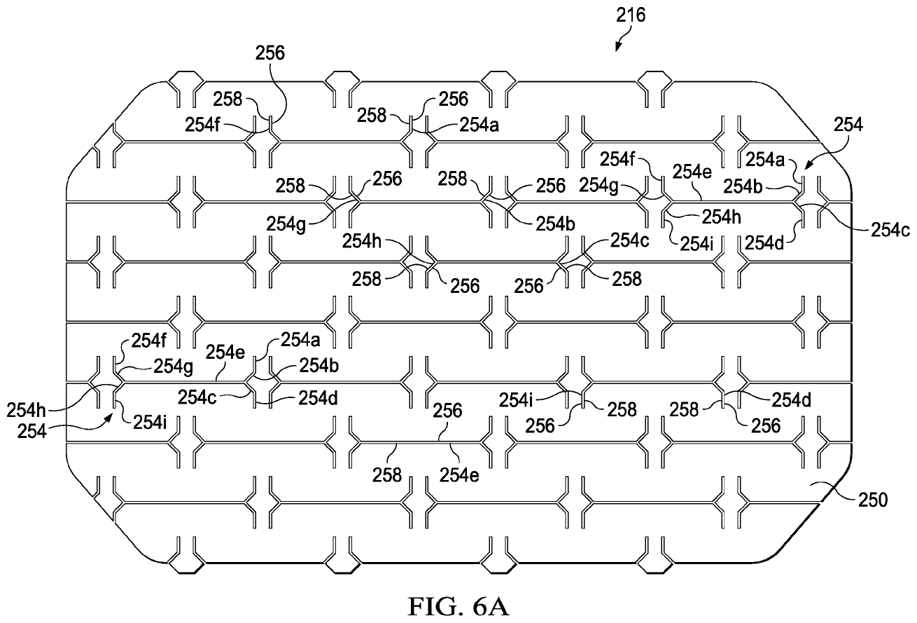

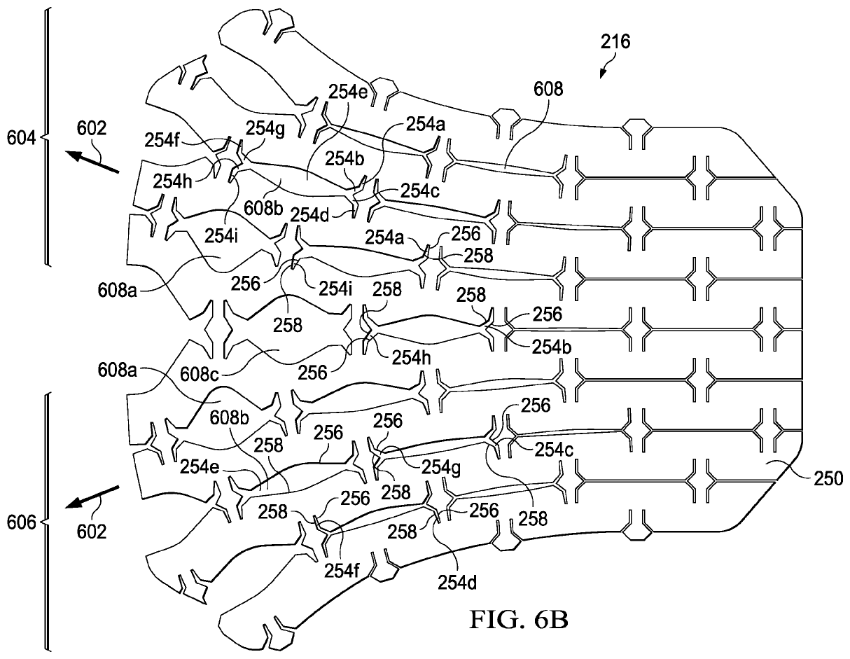

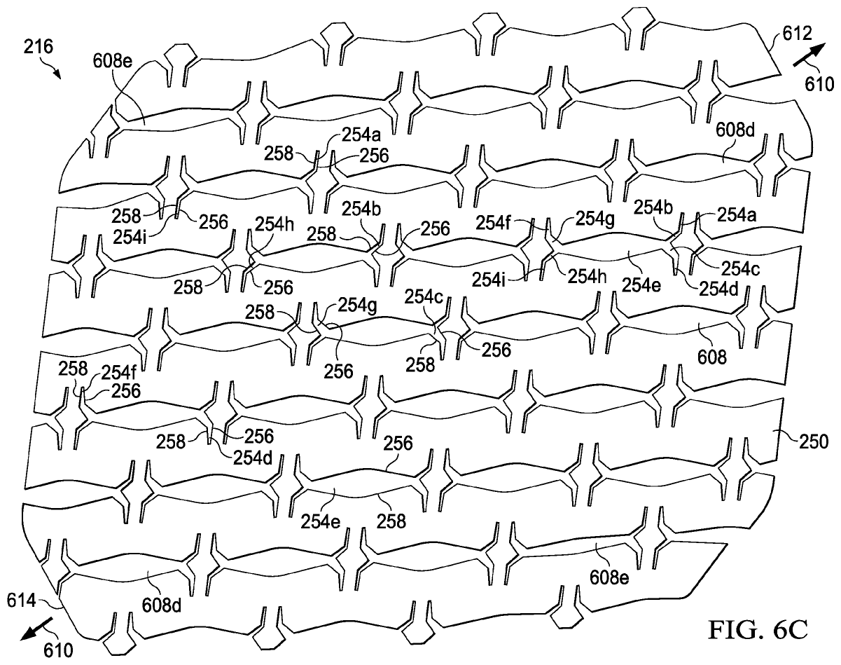

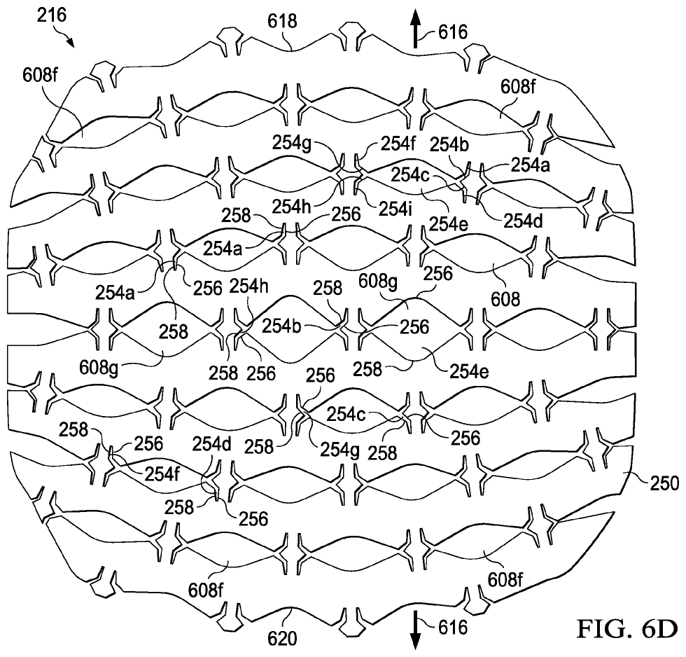

Referring to FIGS. 6A-6D, an embodiment of the manifold 216 is depicted including the slits 254 positioned in different orientations.

FIG. 6A is a top view of the manifold 216 in a resting state. In the resting state, there is no force acting upon the manifold and each slit 254 of the plurality of slits 254 may be in the closed state. In some embodiments, each slit 254 of the plurality of slits 254 may include a first slit 254a, a second slit 254b, a third slit 254c, a fourth slit 254d, a fifth slit 254c, a sixth slit 254f, a seventh slit 254g, an eighth slit 254h, and a ninth slit 254i. Each of the first slit 254a, the second slit 254b, the third slit 254c, the fourth slit 254d, the fifth slit 254e, the sixth slit 254f, the seventh slit 254g, the eighth slit 254h, and the ninth slit 254i may be a linear cut and may include the first side wall 256 and the second side wall 258 that may extend between the first surface 250 and the second surface 252 of the manifold 216.

In an embodiment of the dressing 110 that includes the manifold 216 of FIG. 6A, the plurality of fenestrations 270 of the film layer 214 may have the same size and shape as the plurality of slits 254 of the manifold 216. The plurality of slits 254 of the manifold 216 may align with the plurality of fenestrations 270 of the film layer 214 such that the plurality of slits 254 of the manifold 216 are configured to deform in alignment with the plurality of fenestrations 270 of the film layer 214 when the dressing 110 is exposed to a force such as negative pressure from the negative-pressure source 105.

In other embodiments, the plurality of slits 254 through the manifold 216 may be positioned in another orientation, pattern or length that may be cut through the manifold 216. The plurality of slits 254 may be uniformly distributed across the manifold 216 in some embodiments. In other embodiments, the plurality of slits 254 may be randomly distributed across the manifold 216. In any of the described embodiments, the plurality of slits 254 of the manifold may enable the manifold 216 to be deformed in response to a force that is applied to the dressing 110.

FIG. 6B is a top view of the manifold 216 of FIG. 6A being acted upon by a force 602. The force 602 may be acting on a first half 604 of the manifold 216 and a second half 606 of the manifold 216 such that the first half 604 is being pulled away from the second half 606 and the second half 606 is being pulled away from the first half 604. When acted upon by the force 602, some of the plurality of slits 254 may be deformed from the closed state of FIG. 6A to the open state. Each slit 254 of the plurality of slits 254 may be deformed independently from another of the plurality of slits 254. For example, the plurality of slits proximate to the force 602 may be deformed from the closed state to the open state but the plurality of slits 254 further from the force 602 may not be deformed and may remain in the closed state.

When in the open state, there may be a slit opening 608 defined through the plurality of slits 254 of the manifold 216. In the embodiments of FIGS. 2-5, a slit opening such as the slit opening 608 may be defined between the first side wall 256 and the second side wall 258 when exposed to a force, such as the force 602, as illustrated in FIGS. 6A-6D. As shown in FIGS. 6A and 6B, the slit opening 608 may be formed between at least one of the first slit 254a, the second slit 254b, the third slit 254c, the fourth slit 254d, the fifth slit 254c, the sixth slit 254f, the seventh slit 254g, the eighth slit 254h, and the ninth slit 254i depending the direction and nature of the force applied. For example, the first side wall 256 may be separated from the second side wall 258 in each of the first slit 254a, the second slit 254b, the third slit 254c, the fourth slit 254d, the fifth slit 254c, the sixth slit 254f, the seventh slit 254g, the eighth slit 254h, and the ninth slit 254i when the force applied is substantially uniform or greater than a threshold amount across the surface area of the manifold 216. However, the first side wall 256 may be separated from the second side wall 258 in only some of the first slit 254a, the second slit 254b, the third slit 254c, the fourth slit 254d, the fifth slit 254c, the sixth slit 254f, the seventh slit 254g, the eighth slit 254h, and the ninth slit 254i when the force applied is non-uniform or less than a threshold amount in one or more locations across the surface area of the manifold 216. For example, the first side wall 256 may be separated from the second side wall 258 in the first slit 254a, the second slit 254b, the third slit 254c, the fourth slit 254d, and the fifth slit 254c, but the first side wall 256 may be coupled to the second side wall 258 in the sixth slit 254f, the seventh slit 254g, the eighth slit 254h, and the ninth slit 254i. The first side wall 256 may be separated from the second side wall 258 in other combinations of the first slit 254a, the second slit 254b, the third slit 254c, the fourth slit 254d, the fifth slit 254c, the sixth slit 254f, the seventh slit 254g, the eighth slit 254h, and the ninth slit 254i depending on where the force 602 is acting on the manifold 216.

In some embodiments, the plurality of slits 254 closer to the force 602 may be deformed more than the plurality of slits 254 further from the force 602. For example, a slit opening 608a of the plurality of slits 254 closest to the force 602 may be larger than a slit opening 608b further from the force 602. In some embodiments, there may be a slit opening 608c that is equidistant between the force 602 on the first half 604 and the force 602 on the second half 606. The slit opening 608c may be larger than both the slit opening 608a and the slit opening 608b.

FIG. 6C is a top view of the manifold 216 of FIG. 6A being acted upon by a force 610. The force 610 may be acting on a first corner 612 of the manifold 216 and a second corner 614 of the manifold 216. The first corner 612 may be opposite the second corner 614 and the force 610 may be pulling the first corner 612 away from the second corner 614 while also pulling the second corner 614 away from the first corner 612. When acted upon by the force 610, some of the plurality of slits 254 may be deformed from the closed state of FIG. 6A to the open state. Each slit 254 of the plurality of slits 254 may be deformed independently from another of the plurality of slits 254. For example, the plurality of slits proximate to the force 610 may be deformed from the closed state to the open state to create a slit opening 608d. The plurality of slits further from the force 610 may be deformed less severely such that a slit opening 608c of the plurality of slits 254 further from the force 610 is smaller than the slit opening 608a of the plurality of slits 254 proximate to the force 610.

FIG. 6D is a top view of the manifold 216 of FIG. 6A being acted upon by a force 616. The force 616 may be acting on a first edge 618 of the manifold and a second edge 620 of the manifold 216. The first edge 618 may be opposite the second edge 620 and the force 616 may be pulling the first edge 618 away from the second edge 620 while also pulling the second edge 620 away from the first edge 618. When acted upon by the force 616, some of the plurality of slits 254 may be deformed from the closed state of FIG. 6A to the open state. Each slit 254 of the plurality of slits 254 may be deformed independently from another of the plurality of slits 254. For example, the plurality of slits proximate to the force 616 may be deformed from the closed state to the open state to create a slit opening 608f. The plurality of slits further from the force 610 may be deformed more severely to create a slit opening 608g. The plurality of slits 254 with the slit opening 608g may be equidistant from the force 616 on the first edge 618 and the force 616 of the second edge 620. The plurality of slits 254 with the slit opening 608g may be equally acted upon by the force 616 of the first edge 618 and the force 616 of the second edge 620. Each slit 254 of the plurality of slits 254 closer to the force 616 of the first edge 618 or the second edge 620 may be acted on unevenly by the force 616 on the first edge 618 and the force 616 on the second edge 620 which may result in the slit opening 608f being smaller than the slit opening 608g.

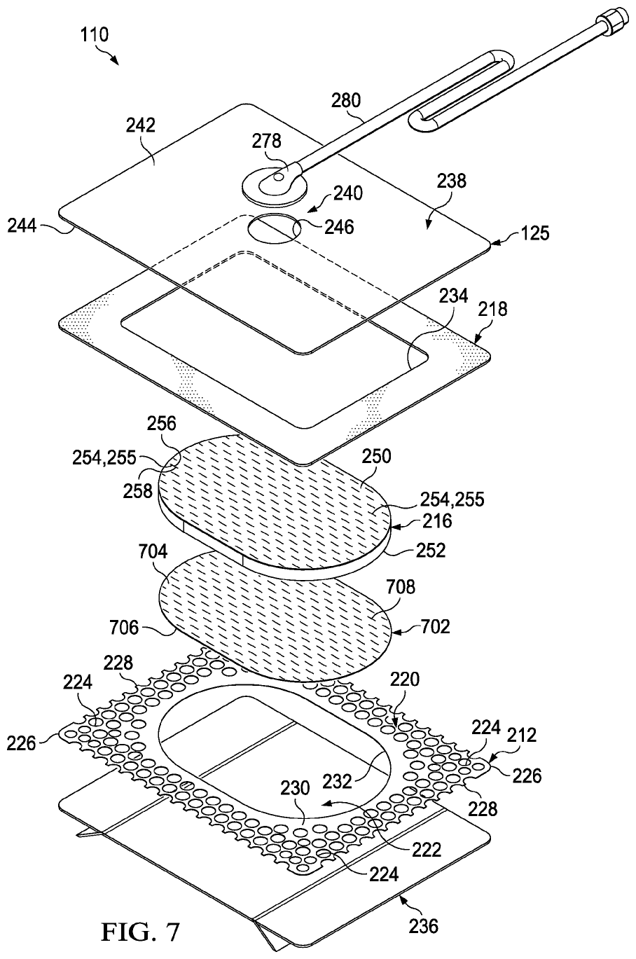

Referring to FIG. 7, another embodiment of the dressing 110 is shown. The dressing interface 278, the conduit 280, the cover 125, the adhesive 218, the manifold 216, the base layer 212, and the release liner 236 may be substantially as described above with reference to FIGS. 2-4. The dressing 110 of FIG. 7 may include a film layer 702 positioned between the manifold 216 and the base layer 212. The film layer 702 may be similar to the central portion 266 of the film layer 214 as described above. In some embodiments, the dressing 110 may further include handling bars similar to handling bars 282 described above with reference to FIG. 3.

The film layer 702 may be substantially the same shape as the manifold 216 and the base layer opening 232. The film layer 702 may contact the tissue site 202 through the base layer opening substantially as described above. The film layer 702 may include a first surface 704, a second surface 706, and a plurality of fenestrations 708. The first surface 704 of the film layer 702 may be configured to couple to the second surface 252 of the manifold 216. Each fenestration 708 of the plurality of fenestrations 708 may be a slit or a cut through the film layer 702 from the first surface 704 of the film layer 702 to the second surface 706 of the film layer 702. The plurality of fenestrations 708 of the film layer 702 may be aligned with the plurality of slits 254 of the manifold 216. The plurality of fenestrations 708 may be configured to deform in concert with the plurality of slits 254 of the manifold 216 when the dressing 110 is exposed to a force such as a bending moment or a negative pressure from the negative-pressure source 105.

The manifold 216 and the film layer 702 may be isolated from the adhesive 218 and may be substantially aligned with the central portion 240 of the cover and the central portion 222 of the base layer 212. The adhesive 218 may be exposed to the tissue surrounding the tissue site 202 through the apertures 224 of the base layer 212 without coming into contact with the film layer 702.

Also described herein is a method for treating a tissue site such as the tissue site 202. The method may include applying the dressing 110 to the tissue site 202, fluidly coupling the negative-pressure source 105 to the dressing 110, and actuating the negative-pressure source 105 to apply negative pressure to the dressing 110. The dressing 110 may include a foam such as the manifold 216 which may include the plurality of slits 254. The dressing 110 may further include the film layer 214. The film layer 214 may be positioned between the foam and the tissue site 202. The film layer 214 may include the plurality of fenestrations 270 configured to align with the plurality of slits 254 of the foam. The plurality of slits 254 of the foam may be deformed in concert with the plurality of fenestrations 270 of the film layer 214 when the negative pressure is applied to the dressing 110 and/or when exposed to another force, such as a bending moment, when the dressing 110 is wrapped around, pressed into, or otherwise conformed to the shape of a particular tissue site.

The systems, apparatuses, and methods described herein may provide significant advantages. For example, the manifold 216 with the plurality of slits 254 may reduce the risk of tissue ingrowth into the dressing 110. In contrast to a conventional aperture or opening, the configuration of the slits 254 prevents portions of the manifold 216 from being sucked through the fenestrations 270 in the film layer 214, while under negative pressure, and into direct contact with a tissue site where tissue ingrowth can occur. Further, the plurality of slits 254 of the manifold 216 may align with the plurality of fenestrations 270 of the film layer 214 to support the removal of thick exudate from the tissue site 202. Further, the plurality of slits 254 of the manifold 216 may create a more even distribution of negative pressure from the negative-pressure source 105 to the tissue site 202 and may support wound healing by breaking the biofilm in the wound bed of the tissue site 202.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the dressing 110, the container 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A dressing for treating a tissue site with negative pressure, the dressing comprising:
a cover comprising a first surface and a second surface;
a foam comprising a first surface, a second surface, and a plurality of slits, the first surface of the foam adjacent to the second surface of the cover, the plurality of slits comprising a first side wall and a second side wall extending between the first surface and the second surface of the foam that are deformable between a closed state and an open state, the first side wall in contact with the second side wall in the closed state, and the first side wall separated from the second side wall in the open state; and
a film layer comprising a first surface, a second surface, and a plurality of fenestrations through the first surface and the second surface that are aligned with the plurality of slits of the foam, the first surface of the film layer adjacent to the second surface of the foam.

2. The dressing of claim 1, wherein the foam is positioned between the cover and the film layer.

3. The dressing of claim 1, wherein the plurality of slits of the foam are configured to deform from the closed state to the open state when the dressing is exposed to the negative pressure, an axial force, or to a bending moment.

4. (canceled)

5. (canceled)

6. The dressing of claim 1, wherein the first side wall is positioned opposite from the second side wall across a slit opening of the plurality of slits.

7. The dressing of claim 1, wherein one or more of the plurality of slits is deformable between the closed state and the open state independently of another of the plurality of slits.

8. The dressing of claim 1, wherein the plurality of fenestrations of the film layer are configured to deform in alignment with the plurality of slits of the foam when the dressing is exposed to the negative pressure.

9. The dressing of claim 1, wherein the plurality of fenestrations of the film layer and the plurality of slits in the foam are aligned in both the open state and the closed state and configured to deform in concert in response to the negative pressure.

10. The dressing of claim 9, wherein the plurality of fenestrations each comprise opposing edges that are positioned flush with the first side wall and the second side wall of the plurality of slits.

11. (canceled)

12. (canceled)

13. The dressing of claim 1, wherein the film layer is coupled to the foam.

14. The dressing of claim 1, further comprising a base layer adjacent to the second surface of the film layer, the base layer comprising a central portion including a base layer opening and a peripheral portion including a plurality of apertures.

15. The dressing of claim 14, wherein the peripheral portion surrounds the central portion, and wherein the plurality of apertures in the peripheral portion are smaller than the base layer opening in the central portion.

16. (canceled)

17. The dressing of claim 14, wherein the base layer opening is a single opening.

18. The dressing of claim 14, wherein the plurality of fenestrations and at least a portion of the film layer are exposed through the base layer opening.

19. The dressing of claim 14, wherein the film layer is configured to contact the tissue site through the base layer opening, and wherein the peripheral portion of the base layer is configured to contact tissue surrounding the tissue site.

20. The dressing of claim 19, wherein the base layer does not contact the tissue site.

21. The dressing of claim 14, wherein the base layer comprises a silicone gel configured to be in contact with tissue around the tissue site.

22. The dressing of claim 14, wherein the film layer comprises a central portion including the plurality of fenestrations aligned with the foam and a peripheral portion extending beyond the foam and including a plurality of perforations surrounding the plurality of fenestrations, wherein the plurality of perforations are larger than the plurality of fenestrations and configured to align with the plurality of apertures of the base layer.

23.-26. (canceled)

27. A system for treating a tissue site with negative pressure, the system comprising:
a dressing according to claim 1; and
a negative-pressure source configured to be fluidly coupled to the dressing.

28. A tissue interface for treating a tissue site with negative pressure, comprising:
a manifold layer comprising a plurality of slits, each of the plurality of slits comprising a first side wall and a second side wall; and
a film layer coupled to a surface of the manifold layer and comprising a plurality of fenestrations, each of the plurality of fenestrations including opposing edges positioned flush with the first side wall and the second side wall of the plurality of slits.

29. A method of treating a tissue site, the method comprising:
applying a dressing to the tissue site, the dressing comprising:
a foam comprising a plurality of slits; and
a film layer positioned between the foam and the tissue site, the film layer comprising a plurality of fenestrations configured to align with the plurality of slits of the foam;
fluidly coupling a negative-pressure source to the dressing; and
actuating the negative-pressure source to apply negative pressure to the dressing, wherein the plurality of slits of the foam are deformed in concert with the plurality of fenestrations of the film layer when the negative pressure is applied to the dressing.

30. (canceled)
